(19) United States
(12) Patent Application Publication
(10) Pub. No.: US 2018/0030483 A1
(43) Pub. Date: Feb. 1, 2018

(54) PROCESS FOR PRODUCTION OF BIO-ALCOHOL

(71) Applicant: DANISCO US INC., Palo Alto, CA (US)

(72) Inventors: Gang DUAN, Shanghai (CN); Vipul GOHEL, Hyderabad (IN); Jayarama K. SHETTY, Pleasanton, CA (US)

(73) Assignee: DANISCO US INC., Palo Alto, CA (US)

(21) Appl. No.: 15/550,713

(22) PCT Filed: Mar. 1, 2016
(86) PCT No.: PCT/US2016/020257

§ 371 Date: Aug. 11, 2017

(30) Foreign Application Priority Data
Mar. 4, 2015 (CN) PCT/CN2015/073620

(51) Int. Cl. C12P 7/06; C12N 1/16

(52) U.S. Cl. CPC C12P 7/06; C12N 1/16

(57) ABSTRACT

A process of enhanced bio-alcohol production from molasses is provided herein. The process comprises contacting molasses with sodium benzoate, potassium sorbate or a mixture thereof followed by yeast fermentation resulting in at least 6% increased alcohol production. Also included within the scope of the present invention is a process for producing co-product containing reduced glycerol.

### CROSS REFERENCE TO RELATED APPLICATION

This application claims the benefit of International Application No. PCT/CN2015/073620, filed Mar. 4, 2015, and is incorporated by reference in its entirety.

### TECHNICAL FIELD

The present invention relates to a process of bio-alcohol production from molasses fermentation, where the fermentation is facilitated by yeast. The invention particularly relates to utilization of sodium benzoate, potassium sorbate of a mixture thereof in molasses fermentation processes.

### BACKGROUND OF THE INVENTION

Bio-alcohols are receiving increasing attention worldwide as a result of high energy prices, the growing focus on clean energy technologies and concern about global climate change. Also, bio-alcohols are important renewable fuels contributing to the reduction of negative environmental impacts generated by the worldwide utilization of the fossil fuels.

Bio-alcohols can be produced from renewable resources such as raw materials containing sugars such as molasses, corn stover, corncob, bagass, rice straw, sugarcane, sugar beet etc; starch such as grains, potato, corn etc; and cellulose such as lignocellulosic biomass comprising cellulose, hemicellulose, and lignin.

Molasses are the waste thick by-product obtained from sugar industries during preparation of sucrose by repeated evaporation, crystallization, and centrifugation of sugarcane or sugar beet juice etc. They are cheap raw materials and readily available for fermentation. About 48% of the total molasses was produced in Asia, and the major share of that was produced in India, China, and Thailand. The molasses produced from cane and beet has a similar sugar composition. Both types of molasses contain both fermentable and non-fermentable sugars. However, beet molasses contains a lower concentration of fermentable sugars and a higher concentration of non-fermentable sugars than cane molasses. Sugarcane molasses is a dark viscous fluid with pH value of 5 and very rich in nutrients required by most microorganisms. It is composed of 68.36% sucrose, 18.50% glucose, and 13.14% maltose. Sugarcane molasses is rich in fermentable sugars approximately 40-55% (wt %) and non-fermentable sugars recorded approximately 5% (wt %). There are various sources of molasses such as sugar cane, sugar beet, starch, maize, sweet sorghum and citrus juice etc. that are rich source of carbohydrates. Such carbohydrates can be consumed by a large number of microorganisms to produce various products.

Production of alcohol from molasses by industrial microorganisms such as yeast (Saccharomyces cerevisiae) is of great commercial importance. Most of the readily available sugars such as sucrose, glucose and fructose in molasses can be consumed by the yeast during fermentation. The fermentation efficiency or the carbon conversion into end products during yeast fermentation depends on several factors, i.e. pH, and temperature, dry solids content, organic acid and most importantly glycerol. The production of unproductive yeast, often referred as wild yeast is undesirable because it produces only biomass at the cost of end product. State of the art describes various processes for production of bio-alcohol. However, efforts to enhance alcohol production have fallen short of expectation. Thus, there is an immediate need to develop novel cost effective methods to improve the efficiency of alcohol yield and reduce unproductive yeast without affecting the alcohol yield.

The present invention addresses this concern and provides a process for enhanced alcohol yield with reduced unproductive yeast production using at least one chemical agent during fermentation.

### SUMMARY OF THE INVENTION

An aspect of the invention is to provide a process for enhanced production of alcohol by fermentation of molasses, comprising contacting the molasses with at least one organic acid salt ranging from 10 ppm to 1000 ppm and fermenting said molasses in the presence of a fermenting organism, wherein the organic acid salt is sodium benzoate or potassium sorbate or a mixture thereof.

Another aspect of the invention is to provide a composition comprising yeast enriched with at least one organic acid salt or a mixture thereof.

Another aspect of the invention is to provide a process for enhanced alcohol production from molasses fermentation, comprising fermenting said molasses in the presence of said composition comprising yeast enriched with at least one organic acid salt or a mixture thereof with or without additional yeast.

### BRIEF DESCRIPTION OF THE ACCOMPANYING DRAWINGS

The accompanying drawings, which are incorporated herein and form part of the specification, illustrate the present invention and, together with the description, further serve to explain the principles of the invention and to enable a person skilled in the relevant art(s) to make and use the invention. In the drawings, like reference numbers, letters, or renderings indicate identical or functionally similar elements.

FIG. 1 shows effect of potassium sorbate on ethanol and glycerol production in YEGM medium.

FIG. 2 shows effect of potassium sorbate on yeast growth in YEGM medium.

FIG. 3 shows effect of potassium sorbate on ethanol and glycerol production in molasses fermentation.

FIG. 4 shows effect of potassium sorbate on acetic acid production in molasses fermentation.

FIG. 5 shows effect of sodium benzoate on ethanol and glycerol production in YEGM medium.

FIG. 6 shows effect of sodium benzoate on yeast growth in YEGM medium.

FIG. 7 shows effect of sodium benzoate on ethanol and glycerol production in molasses fermentation.

FIG. 8 shows combine effect of potassium sorbate and sodium benzoate on ethanol and glycerol production in molasses fermentation.

FIG. 9 shows effect of potassium sorbate enriched cream yeast on ethanol and glycerol production in molasses fermentation.

FIG. 10 shows effect of potassium sorbate enriched dry yeast on ethanol and glycerol production in molasses fermentation.

FIG. 11 shows effect of sodium benzoate enriched cream yeast on ethanol and glycerol production in molasses fermentation.

FIG. 12 shows effect of sodium benzoate enriched dry yeast on ethanol and glycerol production in molasses fermentation.

FIG. 13 shows effect of potassium sorbate enriched cream yeast on acetic acid production in molasses fermentation.

FIG. 14 shows effect of potassium sorbate enriched dry yeast on acetic acid production in molasses fermentation.

FIG. 15 shows effect of sodium benzoate enriched cream yeast on acetic acid production in molasses fermentation.

FIG. 16 shows effect of sodium benzoate enriched dry yeast on acetic acid production in molasses fermentation.

### DETAILS DESCRIPTION OF THE INVENTION

Unless otherwise defined, all technical and scientific terms used in the present specification and the appended claims have the same meaning as commonly understood by one of ordinary skill in the art to which this invention belongs.

The following definitions are provided as an aid to understand the present disclosure.

It is to be understood that the disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. As used herein, where the indefinite article “a” or “an” is used with respect to a statement or description of the presence of a step in a process disclosed herein, unless the statement or description explicitly provides to the contrary, the use of such indefinite article is not intended to limit the presence of the step in the process to one in number.

Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the disclosure pertains. Although any methods and materials similar or equivalent to those described herein can be used for testing of the subject matter recited in the current disclosure, the preferred materials and methods are described herein. In describing and claiming the subject matter of the current disclosure, the following terminology will be used in accordance with the definitions set out below.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

When a composition, a process, a structure, or a portion of a composition, a process, or a structure, is described herein using an open-ended term such as “comprising,” unless otherwise stated the description also includes an embodiment that “consists essentially of” or “consists of” the elements of the composition, the process, the structure, or the portion of the composition, the process, or the structure.

Further in this connection, certain features of the invention which are, for clarity, described herein in the context of separate embodiments, may also be provided in combination in a single embodiment. Conversely, various features of the invention that are, for brevity, described in the context of a single embodiment, may also be provided separately or in any sub-combination.

The term “optionally” is used herein to mean “is provided in some embodiments and not provided in other embodiments”. Any particular embodiment of the invention may include a plurality of “optional” features unless such features conflict.

Throughout this application, various embodiments of the invention may be presented in a range format. It should be understood that the description in range format is merely for convenience and should not be construed as a limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “in the range/ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” or “process” refers to manners, means, techniques and procedures for accomplishing a given task including, but not limited to, those manners, means, techniques and procedures either known to, or readily developed from known manners, means, techniques and procedures by practitioners of the chemical, biological, microbiological and biochemical arts.

The term “contacting” refers to the placing of the respective enzymes in sufficiently close proximity to the respective substrate to enable the enzymes to convert the substrate to the end product. Those skilled in the art will recognize that mixing solutions of the enzyme with the respective substrates can effect contacting.

The term “fermentation products” or “fermentation yield” includes a product produced by a fermentation process. The fermentation products include alcohol (e.g. ethanol, methanol, propanol and butanol), organic acids such as citric acid, lactic succinic acid, acetic acid and gluconic acid), and amino acids such as glutamic acid, tryptophan, threonine, and methionine.

The term “fermenting organism” refers to any organism, including bacterial, fungal and yeast, suitable for producing a desired fermentation end products like alcohol such as ethanol and butanol, amino acids, organic acids such as lactic acid, citric acid, succinic acid, monosodium glutamate, and 1-3 propane diol.

Example of fermenting organisms includes fungal organisms such as yeast. Preferred yeast includes, but is not limited to strains of the genus Saccharomyces, Pichia, Candida. Dekkera, Hanseniaspora, Pseudozyma, Sacharromycodes, Zygosaccharomyces, Zygosaccharomyces, Zygoascus spp, Issatchenkia, Williopsis, Torulaspora, Debaryomyces, Zygosaccharomyces, Brettanomyces and Brettanomyces.

The term “molasses” refers to a concentrate from mother liquor from sugar crystallization process. Examples of molasses source include but are not limited to sugar cane, sugar beet, starch, maize, sweet sorghum, sugar cane juice, and citrus juice.

The term “sodium benzoate enriched yeast” refers to yeast produced by contacting yeast such as Saccharomyces cerevisiae in a sodium benzoate rich media.

The term “Potassium sorbate enriched yeast” refers to yeast produced by contacting yeast such as Saccharomyces cerevisiae in a potassium sorbate rich media.

The term “Potassium sorbate and sodium benozate enriched yeast” refers to yeast produced by contacting yeast such as Saccharomyces cerevisiae in a potassium sorbate and sodium benozate rich media.

It is to be understood that both the foregoing general description and the following detailed description are exemplary and explanatory only and are not restrictive of the compositions, films, and methods described herein. In this application, the use of the singular includes the plural unless specifically state otherwise. Also, the use of “or” means “and/or” unless state otherwise.

The following discussion is directed to the preferred embodiments of the present invention only, and nothing within the following disclosure is intended to limit the overall scope of this invention. The scope of the present invention is to be defined solely by the claims, as presented at the end of this specification.

The present invention provides a process for enhanced alcohol production during molasses using organic acid salts in fermentation medium or using organic acid enriched yeast in molasses fermentation. The process disclosed herein comprises contacting molasses with organic acid salts followed by yeast fermentation producing increased yield of alcohol and reduced glycerol and lower yeast count. Also included within the scope of the present invention results in increased fermentation product yield, i.e. alcohol. The increase in the fermentation yield is accomplished by the addition of organic compounds containing carboxylic acid with a non-polar aliphatic or aromatic group. In a preferred embodiment, the organic acid may be presented by the formula:

R—COO⁻

Where R is H or an aliphatic or aromatic group containing from 1 to 14 carbon atoms. More specifically R can be methyl, ethyl, n- or isopropyl, n- or iso-butyl, phenyl or substituted phenyl. Preferably, R can be aromatic ring with carboxylic group such as benzoic, pheny acetic and pheny propionic acids. The carboxylate ion containing organic acid can be introduced to the aqueous medium of yeast fermentation containing starch or fermentable sugars in the form of carboxylic acid or its' salts, e.g. a sodium, potassium or calcium salt of the corresponding carboxylic acid.

Preferred bacterial fermenting organisms include strains of Escherichia, strains of Zymomonas and Klebsialla. Preferably any strain capable of producing alcohol, i.e. ethanol, butanol, methanol, propanol etc.

Surprisingly, the inventors found that alcohol yield can be increased by at least 6 to 8% with the use of sodium benzoate, potassium sorbate or a mixture thereof during molasses fermentation.

In one embodiment of the invention there is provided a process for enhanced production of alcohol by fermentation of molasses, comprising contacting the molasses with at least one organic acid salt ranging from 10 ppm to 1000 ppm and fermenting said molasses in the presence of a fermenting organism, wherein the organic acid salt is sodium benzoate, potassium sorbate or a mixture thereof.

Another embodiment of the invention provides a process for enhanced production of alcohol by fermentation of molasses, comprising contacting the molasses with at least one organic acid salt ranging from 10 ppm to 1000 ppm at a temperature ranging from 15° C. to 70° C., at a pH from 3.0 to 7.0, and fermenting said molasses at a temperature ranging from 15° C. to 40° C. at a pH from about 3.0 to 7.0 in the presence of a fermenting organism, wherein the organic acid salt is sodium benzoate, potassium sorbate or a mixture thereof.

Another embodiment of the invention relates to alcohol, wherein the alcohol is selected from a group consisting of ethanol, butanol, methanol, and propanol.

Yet another embodiment of the invention relates to fermenting organism for molasses fermentation in presence of one or more organic salt, wherein said fermenting organism is recombinant organism.

Further embodiment of the invention relates to fermenting organism for molasses fermentation in presence of one or more organic salt, wherein said fermenting organism is yeast. Another embodiment of the invention relates to fermenting organism for molasses fermentation in presence of one or more organic salt, wherein said fermenting organism recombinant yeast.

Another embodiment of the invention relates to examples of yeast including, but is not limited to Saccharomyces spp, Saccharomyces cerevisiae, Candida spp, Pichia spp, Dekkera spp, Hanseniaspora spp, Pseudozyma spp, Sacharromycodes spp, Zygosaccharomyces spp, Zygosaccharomyces rouxii, Zygoascus spp, Issatchenkia spp, Williopsis spp, Torulaspora delbrueckii, Debaryomyces hansenii, Zygosaccharomyces bailii, Brettanomyces spp. and Brettanomyces bruxellensis.

Another embodiment of the invention provides a process for enhanced production of alcohol by fermentation of molasses, comprising contacting the molasses with at least one organic acid salt ranging from 10 ppm to 1000 ppm and fermenting said molasses in the presence of a fermenting organism, wherein said contacting and fermenting occur simultaneously, wherein the organic acid salt is sodium benzoate, potassium sorbate or a mixture thereof.

Another embodiment of the invention provides a process for enhanced production of alcohol by fermentation of molasses, comprising contacting the molasses with at least one organic acid salt ranging from 10 ppm to 1000 ppm and fermenting said molasses in the presence of a fermenting organism, wherein said contacting occurs as a pre-treatment, wherein the organic acid salt is sodium benzoate, potassium sorbate or a mixture thereof.

Further embodiment of the invention provides a process for enhanced alcohol production from molasses fermentation, comprising fermenting the molasses in the presence of a composition comprising yeast enriched with at least one organic acid salt with or without additional yeast.

Further embodiment of the invention provides a process for enhanced alcohol production from molasses fermentation, comprising fermenting the molasses at a temperature ranging between 15° C. to 40° C., at a pH from about 3.0 to 7.0 in the presence of a composition comprising yeast enriched with at least one organic acid salt with or without additional yeast, wherein the organic acid salt is sodium benzoate, potassium sorbate or a mixture thereof.

Another embodiment of the invention relates to a molasses obtained from a source selected from a group consisting of sugar cane, sugar beet, starch, maize, sweet sorghum, sugar cane juice, and citrus juice.

Another embodiment of the invention relates to a process for enhanced production of alcohol by fermentation of molasses, comprising contacting the molasses with at least one organic acid salt ranging from 10 ppm to 1000 ppm and fermenting said molasses in the presence of a fermenting organism, wherein said process yields at least 6% more alcohol as compared with molasses fermentation carried out without the use of organic salt, wherein the organic acid salt is sodium benzoate, potassium sorbate or a mixture thereof.

Another embodiment of the invention relates to a process for enhanced alcohol production from molasses fermentation, comprising fermenting the molasses in the presence of a composition comprising yeast enriched with at least one organic acid salt with or without additional yeast, wherein said process yields at least 6% more alcohol as compared with molasses fermentation carried out without using the composition, wherein the organic acid salt is sodium benzoate, potassium sorbate or a mixture thereof.

The following Examples are offered by way of illustration, not limitation.

All publications, patents, and patent applications mentioned in this specification are indicative of the level of those skilled in the art to which this invention pertains, and are herein incorporated by reference to the same extent as if each individual publication, patent, or patent application was specifically and individually indicated to be incorporated by reference.

### EXAMPLES

The methods disclosed herein are illustrated in the following examples. From the above discussion and these examples, one skilled in the art can ascertain the various embodiments of this invention, and without departing from the spirit and scope thereof, can make various changes and modifications of the methods and compositions disclosed herein to adapt it to various uses and conditions.

The following additional abbreviations are used: “C” is Celsius, “μ” is microliter, “mL” is milliliter, “M” is molar, “mM” is milimolar, “mm” is milimeter, “nm” is nanometer, “μ” is micron, “min” is minute(s), “g” is gram(s), “mg” is milligram(s), “v/v” is volume by volume, “w/w” is weight by weight, “w/v” is weight by volume, “wt” is weight, “wt %” means weight percent, “rpm” is rotation per minute, “Temp” is temperature, “OD” mean optical density, “MW” means average molecular weight and “hr” means hour, “ADY” means active dry yeast.

### Materials

The following materials were used in the experiments. All commercial reagents were used as received unless otherwise noted.

### Feedstock

Sugarcane molasses is used as a carbon source for fermentation studies. The molasses was stored at 4° C. to 25° C. until use.

### Yeast

Saccharomyces cerevisiae, alcohol dry yeast commercially available from Fali Yeast, AB Mauri was used.

### Media

Yeast Extract Glucose media (YEGM): YEGM was prepared by dissolving 0.75 g Yeast Extract and 10 g dextrose dissolved in 100 ml tap water with pH in range 6.8±2. The media was then sterilized at 121° C. for 20 min.

Molasses Medium: The media was prepared by diluting 333 g molasses with water to final weight of 1000 g (w/w). To this 400 mg of urea was added as nitrogen source.

### Microorganism and Inoculum Preparation

Saccharomyces cerevisiae was used in the form of active dry yeast at a concentration of 3% w/v or was pre-cultured by adding 6 g of active dry yeast into 100 ml YEGM in 250 ml Erlenmeyer's flask. The cultures were incubated at 32° C.±2° C. with mixing at 100 to 200 rpm to a final OD660 of 100±10. This culture was subsequently used as an inoculum for further fermentation studies at a concentration of 6% v/v.

### Analytical Method

### Sugar Composition, Ethanol, and Glycerol Content Determination by HPLC Method:

The composition of the reaction products was measured by high pressure liquid chromatographic method (Agilent Isocratic system 1200, USA with RI detector) by injecting 20 μL of appropriately diluted samples onto a Aminex Column HPX-87H (catalogue number 1250140, Bio-Rad) maintained at 60° C. using 0.01N sulphuric acid as a mobile phase at a flow rate of 0.7 mL/min. The detection was carried out using Refractive index detector (RID).

### Determination of Organic Acid Salts by HPLC Method:

The Potassium Sorbate and Sodium Benzoate in the reaction mixture was quantified by using Waters Acquity H Class Bio System by injecting 5 μL of appropriately diluted samples onto a Acquity UPLC BEH C18 column (100 mm×2.1 mm×1.7μ; Part No—186002352) maintained at 50° C. using mobile phase comprising of 0.05% Acetic acid in 5 mM Ammonium Acetate and Acetonitrile at a flow rate of 0.5 ml/min using a gradient programming as given below. The detection was carried out at 225 nm using UV detector.

### Yeast Biomass Estimation:

The yeast biomass was measured using UV-Vis spectrophotometers (Shimadzu UV-1800) by diluting with YEGM to an OD660 in range of 0.2 to 0.8 with Milli-Q water.

### Example 1

### Effect of Potassium Sorbate on Yeast Fermentation

### YEGM

Batch fermentation was initiated by addition of potassium sorbate at a concentration ranging from 0 to 1000 ppm followed by 6 ml of yeast inoculum with OD (660 nm) of 100±10 into 100 ml YEGM in 250 mL Erlenmeyer flasks. Fermentation was carried out at 32° C.±2° C. in incubator shaker at 100-200 rpm. The fermentation was carried out until glucose value observed was less than 0.1% w/v. Thereafter, the samples were analysed for alcohol and glycerol profile by HPLC and yeast biomass by UV Spectrophotometer.

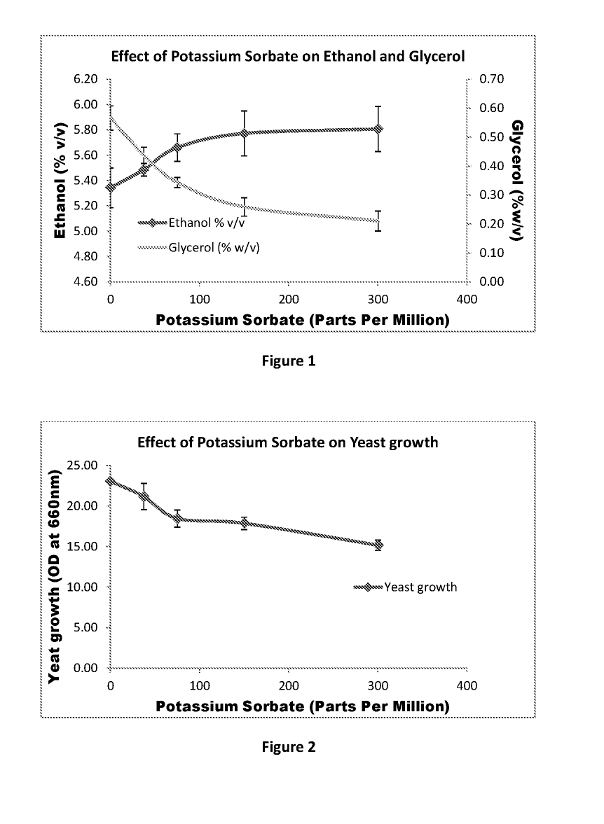

It was observed that increasing concentration of potassium sorbate results in increased ethanol production at least by 6% and reduced glycerol yield (FIG. 1) and reduced yeast growth (FIG. 2).

### Molasses

Batch fermentation was initiated by addition of potassium sorbate at a concentration ranging from 0 to 1000 ppm followed by 3 g of active dry yeast into 100 ml molasses media in 250 mL Erlenmeyer flasks. Fermentation was carried out at 32° C.±2° C. in incubator shaker at 100-200 rpm. The fermentation was carried out until glucose value observed was less than 0.1% w/v. Thereafter, the samples were analysed for alcohol, organic acids, and glycerol profile by HPLC.

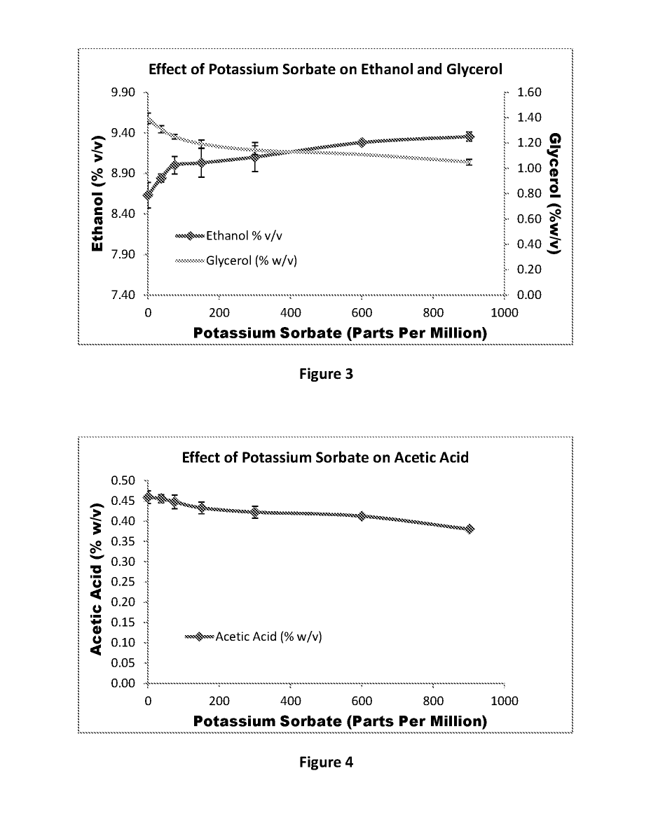

It was observed that increasing concentration of potassium sorbate in molasses fermentation results in increased ethanol production and reduced glycerol and acetic acid yield (FIG. 3-4).

### Example 2

### Effect of Sodium Benzoate on Yeast Fermentation

### YEGM

Batch fermentation was initiated by addition of sodium benzoate at a concentration ranging from 0 to 1000 ppm followed by 6 ml of yeast inoculum with OD (660 nm) of 100±10 into 100 ml YEGM in 250 mL Erlenmeyer flasks. Fermentation was carried out at 32° C.±2° C. in incubator shaker at 100 to 200 rpm. The fermentation was carried out until glucose value observed was less than 0.1% w/v. Thereafter, the samples were analysed for alcohol and glycerol profile by HPLC and yeast biomass by UV Spectrophotometer.

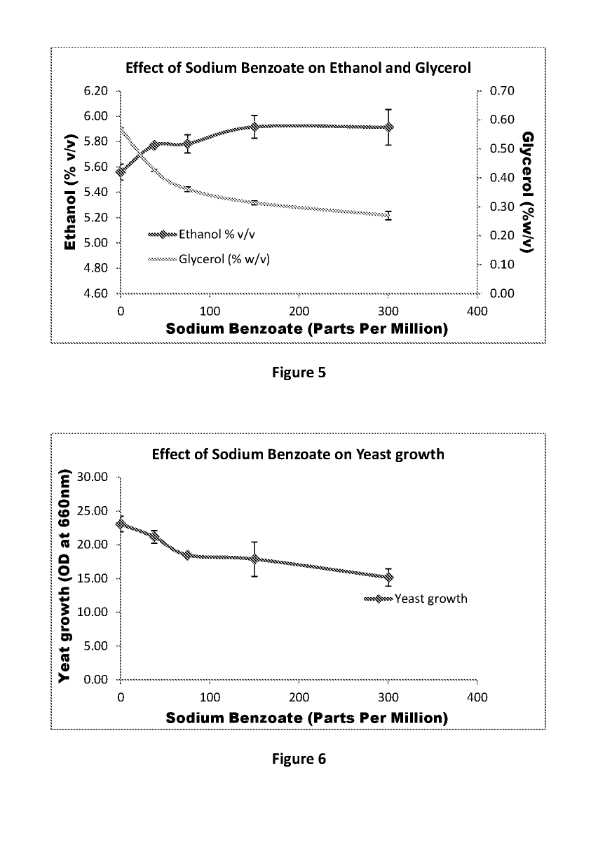

It was observed that increasing concentration of sodium benzoate results in increased ethanol production at least by 6% and reduced glycerol yield (FIG. 5) and reduced yeast growth (FIG. 6).

### Molasses

Batch fermentation was initiated by addition of sodium benzoate at a concentration ranging from 0 to 1000 ppm followed by 3 g of active dry yeast into 100 ml molasses media in 250 mL Erlenmeyer flasks. Fermentation was carried out in incubator shaker at 32° C.±2° C. at 100 to 200 rpm. Thereafter, the samples were analysed for alcohol, organic acids, and glycerol profile by HPLC.

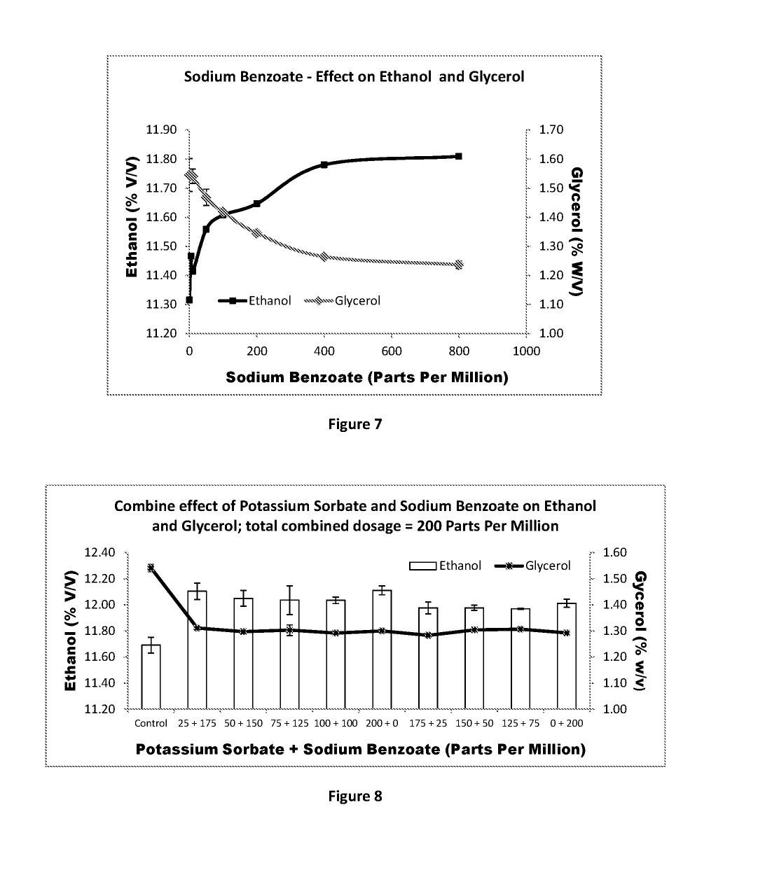

It was observed that increasing concentration of sodium benzoate in molasses fermentation results in increased ethanol production and reduced glycerol yield (FIG. 7).

### Example 3

### Effect of Mixture of Potassium Sorbate and Sodium Benzoate on Yeast Fermentation in Molasses

Batch fermentation was initiated by mixing potassium sorbate (0 to 1000 ppm), sodium benzoate (0 to 1000 ppm) and active dry yeast (3 g) into 100 ml molasses media in 250 mL Erlenmeyer flasks. Fermentation was carried out at 32° C.±2° C. in incubator shaker at 100 to 200 rpm. The fermentation was carried out until glucose value observed was less than 0.1% w/v. Thereafter, the samples were analysed for alcohol, organic acids, and glycerol profile by HPLC.

It was observed that any combinations of potassium sorbate and sodium benzoate alone or in any combination in molasses fermentation results in increased ethanol production and reduced glycerol yield (FIG. 8).

### Example 4

### Enrichment of Yeast with Organic Acids Salts

Yeast Enrichment with Potassium Sorbate

A suspension solution comprising 30% w/w dry yeast, potassium sorbate (0 to 50,000 ppm), and MES (2-(N-morpholino) ethanesulfonic acid) hydrate medium (0.1 M) was prepared and pH of the solution was adjusted to 5.5. The suspension was incubated at 32° C.±2° C. in incubator shaker at 100 to 200 rpm for 2 hrs and centrifuged at 3500 rpm at 25° C. for 10 minutes to obtain potassium sorbate enriched yeast in the form of yeast pellet.

Yeast Enrichment with Sodium Benzoate

A suspension solution comprising 30% w/w dry yeast, sodium benzoate (0 to 50,000 ppm), and MES (2-(N-morpholino)ethanesulfonic acid) hydrate medium (0.1 M) was prepared and pH of the solution was adjusted to 5.5. The suspension was incubated at 32° C.±2° C. in incubator shaker at 100 to 200 rpm for 2 hrs and centrifuged at 3500 rpm at 25° C. for 10 minutes to obtain sodium benzoate enriched yeast in the form of yeast pellet.

Yeast Enrichment with Mixture of Potassium Sorbate and Sodium Benzoate

A suspension solution comprising 30% w/w dry yeast, potassium sorbate (0 to 50,000 ppm) and sodium benzoate (0 to 50,000 ppm), and MES (2-(N-morpholino) ethanesulfonic acid) hydrate medium (0.1 M) was prepared and pH of the solution was adjusted to 5.5. The suspension was incubated at 32° C.±2° C. in incubator shaker at 100 to 200 rpm for 2 hrs and centrifuged at 3500 rpm at 25° C. for 10 minutes to obtain yeast enriched with potassium sorbate and sodium benzoate in the form of yeast pellet.

### Example 5

### Organic Acid Salt Enriched Yeast Formulation

The organic acid salt enriched yeast as described in Example 4 can be used in various forms such as cream yeast, washed cream yeast, yeast cake, and dry yeast.

Cream yeast was prepared by re-suspending the enriched yeast pellet as described in Example 4 in water.

Washed cream yeast was prepared by washing the enriched yeast as described in Example 4 with de-ionized water in centrifuge at 3500 rpm at 25° C. for 10 minutes and re-suspending the yeast pellet in water.

Dry yeast was prepared by extruding the enriched yeast as described in Example 4 as a 2 mm tube and drying. The dried yeast was re-suspended in de-ionized water (30%).

### Example 6

### Molasses Fermentation with Organic Acid Salt Enriched Yeast

Batch fermentation was initiated by addition of enriched yeast as prepared in Example 4 in the form of cream, cake, or active dry at a concentration of 3 g active dry yeast into 100 ml molasses media in 250 mL Erlenmeyer flasks. Fermentation was carried out at 32° C.±2° C. in incubator shaker at 100-200 rpm. The fermentation was carried out until glucose value observed was less than 0.1% w/v. Thereafter, the samples were analysed for alcohol, organic acids, and glycerol profile by HPLC.

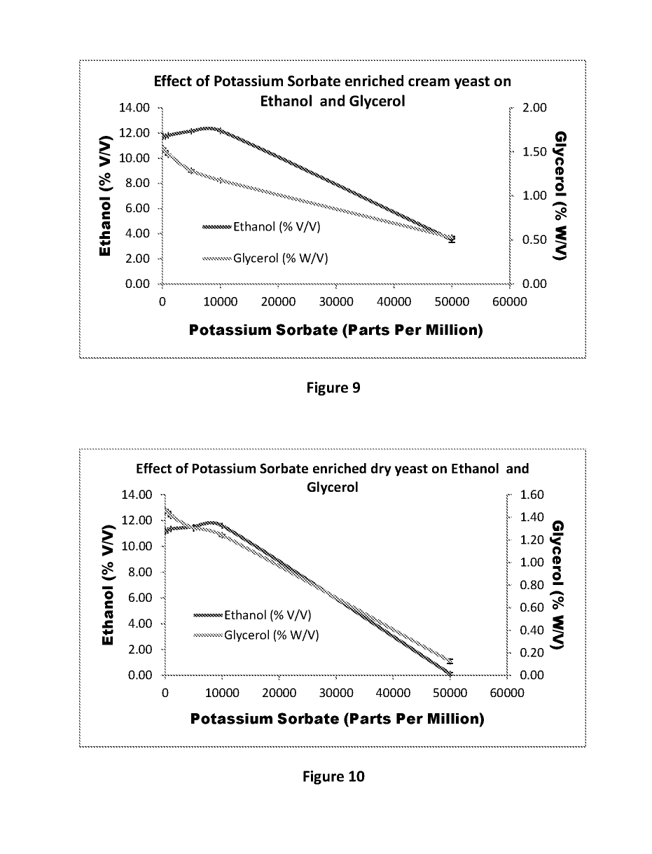

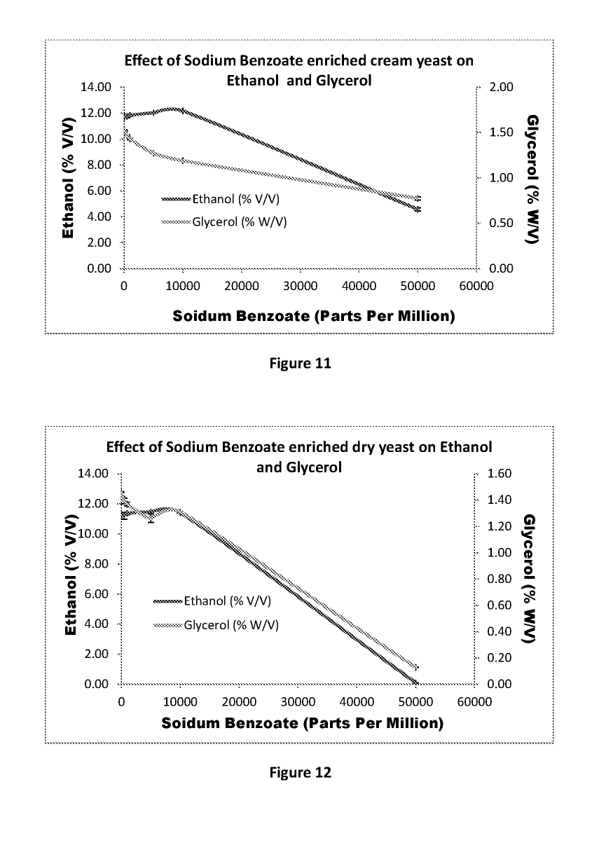

It was observed that potassium sorbate (FIG. 9, 10) or sodium benzoate (FIG. 11, 12) enriched cream or active dry yeast in molasses fermentation results in increased in ethanol production and reduced glycerol yield until 10,000 ppm.

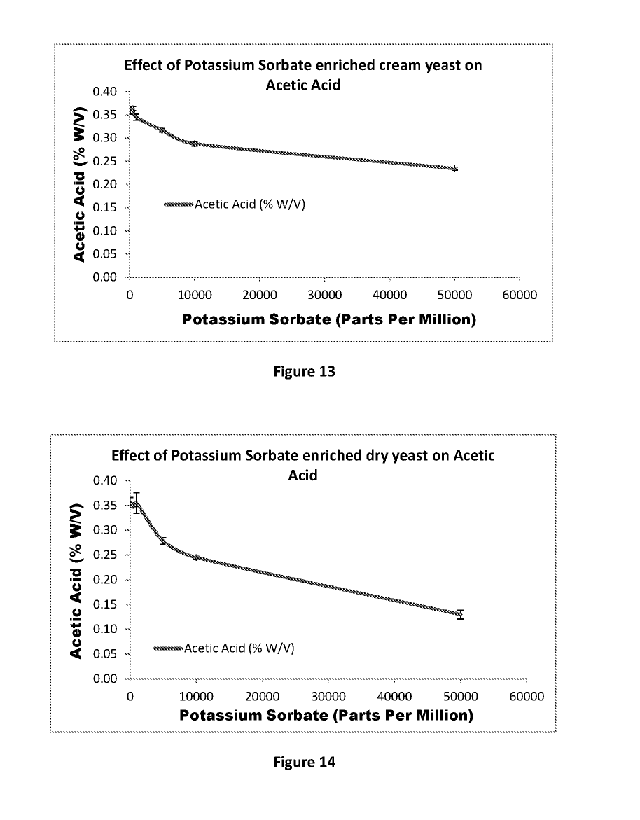

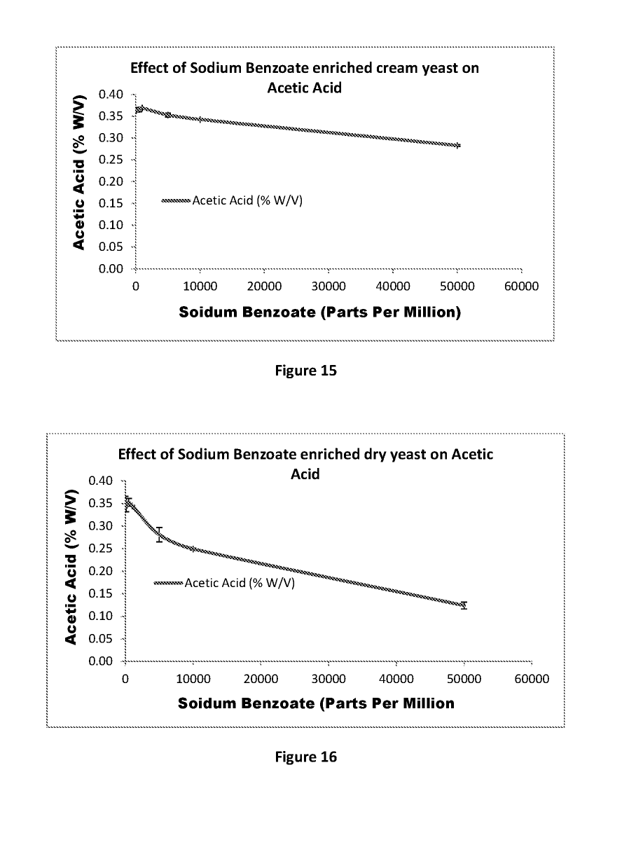

It was observed that potassium sorbate (FIG. 13, 14) or sodium benzoate (FIG. 15, 16) enriched cream or active dry yeast in molasses fermentation results in reduced acetic acid yield.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the appended claims.

1. A process for enhanced production of alcohol by fermentation of molasses, comprising contacting the molasses with at least one organic acid salt ranging from 10 ppm to 1000 ppm and fermenting said molasses in the presence of a fermenting organism, wherein said organic acid salt is sodium benzoate, or potassium sorbate, wherein said contacting occurs as a pre-treatment.

2. The process of claim 1, wherein said alcohol is selected from a group consisting of ethanol, butanol, methanol, and propanol.

3. The process of claim 1, wherein said fermenting organism is a recombinant organism.

4. The process of claim 1, wherein said fermenting organism is a yeast.

5. The process of claim 1, wherein said yeast is a recombinant yeast.

6. The process of claim 5, wherein said yeast is a Saccharomyces sp., Saccharomyces cerevisiae, Candida sp., Pichia sp., Dekkera sp., Hanseniaspora sp., Pseudozyma sp., Sacharromycodes sp., Zygosaccharomyces sp., Zygosaccharomyces rouxii, Zygoascus sp., Issatchenkia sp., Williopsis sp., Torulaspora delbrueckii, Debaryomyces hansenii, Zygosaccharomyces bailii, Brettanomyces sp. and Brettanomyces bruxellensis.

7. (canceled)

8. A composition comprising yeast enriched with at least one organic acid salt or a mixture thereof.

9. The composition as claimed in claim 8, wherein said organic acid salt is sodium benzoate or potassium sorbate or a mixture thereof.

10. The composition as claimed in claim 8, wherein said composition is in form of yeast cake, cream or dry yeast.

11. The composition as claimed in claim 8, wherein said yeast is recombinant or non-recombinant yeast.

12. The composition as claimed in claim 10, wherein said yeast is Saccharomyces cerevisiae.

13. A process for enhanced alcohol production from molasses fermentation, comprising fermenting said molasses in the presence of said composition as claimed in claim 9 with or without additional yeast.

14. The process as claimed in claim 13, wherein said alcohol is selected from a group consisting of ethanol, butanol, methanol and propanol.

15. The process of claim 1, wherein said molasses is obtained from a source selected from a group consisting of sugar cane, sugar beet, starch, maize, sweet sorghum, sugar cane juice, and citrus juice.

16. The process of claim 1, wherein said process yields at least 6% more alcohol as compared with molasses fermentation carried out without using organic salts.

17. The process of claim 13, wherein said molasses is obtained from a source selected from a group consisting of sugar cane, sugar beet, starch, maize, sweet sorghum, sugar cane juice, and citrus juice.

18. The process of claim 13, wherein said process yields at least 6% more alcohol as compared with molasses fermentation carried out without using organic salts.
